(19) United States
(12) Patent Application Publication
(10) Pub. No.: US 2022/0275453 A1
(43) Pub. Date: Sep. 1, 2022

(54) IN VITRO METHOD FOR THE DIAGNOSIS OR PROGNOSIS OF COLORECTAL CANCER OR A PRECANCEROUS STAGE THEREOF

(71) Applicants: SERVIZO GALEGO DE SAÚDE, Santiago de Compostela (ES); FUNDACIÓN INSTITUTO DE INVESTIGACIÓN SANITARIA DE SANTIAGO DE COMPOSTELA, Santiago de Compostela (ES)

(72) Inventors: Ángel DÍAZ LAGARES, Santiago de Compostela (ES); Rafael LÓPEZ LÓPEZ, Santiago de Compostela (ES); Ana Belén CRUJEIRAS MARTÍNEZ, Santiago de Compostela (ES); Aitor RODRÍGUEZ CASANOVA, Santiago de Compostela (ES)

(21) Appl. No.: 17/604,182

(22) PCT Filed: Apr. 15, 2020
(86) PCT No.: PCT/EP2020/060517

§ 371 Date: Oct. 15, 2021

(30) Foreign Application Priority Data
Apr. 15, 2019 (EP) 19382290.5

(51) Int. Cl. C12Q 1/6886

(52) U.S. Cl. CPC C12Q 1/6886; C12Q 2600/118; C12Q 2600/154

(57) ABSTRACT

The method of the invention implies the determination of the methylation status of the gene LINC00473 promoter in a liquid biopsy obtained from the patient.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is 370093_404USPC_SEQUENCE_LISTING.txt. The text file is 2.13 KB, was created on May 11, 2022, and is being submitted electronically via EFS-Web.

### BACKGROUND

### Technical Field

The present invention refers to the medical field. Particularly, the present invention refers to an in vitro method for the diagnosis and/or prognosis of colorectal cancer (CRC) and/or a pre-cancerous stage thereof. The method of the invention comprises the determination of the methylation status of the gene LINC00473, preferably in a liquid biopsy obtained from the patient, wherein a higher level of methylation of the gene LINC00473 with respect to an established reference control level, is indicative of the presence of colorectal cancer, a pre-cancerous stage thereof and/or of poor prognosis of the disease.

### Description of the Related Art

CRC, also known as colon cancer, rectal cancer, or bowel cancer, is the development of cancer in the colon or rectum (parts of the large intestine). The vast majority of colorectal cancers are adenocarcinomas. This is because the colon has numerous glands within the tissue. When these glands undergo a number of changes at the genetic level, they proceed in a predictable manner as they move from benign to an invasive, malignant colon cancer. The adenomas of the colon, particularly advanced colorectal adenoma (AA), are a benign version of the malignant adenocarcinomas but still with malignant potential if not removed (they are usually removed because of their tendency to become malignant and to lead to colon cancer).

Screening is an effective way for preventing and decreasing deaths from colorectal cancer and is recommended starting from the age of 50 to 75. The best known and most frequently used screening test for colorectal cancer is called Fecal Immunochemical Test (FIT). FIT detects blood in the stool samples which can be a sign of pre-cancer or cancer. If abnormal results are obtained, usually a colonoscopy is recommended which allows the physician to look at the inside of the colon and rectum to make a diagnosis. During colonoscopy, small polyps may be removed if found. If a large polyp or tumor is found, a biopsy may be performed to check if it is cancerous. The gastroenterologist uses a colonoscopy to find and remove these adenomas and polyps to prevent them from continuing to acquire genetic changes that will lead to an invasive adenocarcinoma.

Although, as explained above, FIT is nowadays used for screening colorectal cancer, it is important to note that FIT offers a low sensitivity for AA which means that most of said kind of patients can be wrongly classified as not having the disease. Consequently, FIT is not able to identify adenomas due to its low sensitivity. Moreover, since FIT uses stool samples, it offers a low compliance. On the other hand, colonoscopy is an invasive technique wherein the most severe complication generally is the gastrointestinal perforation. Moreover, colonoscopy is nowadays a procedure involving anesthesia, and the laxatives which are usually administered during the bowel preparation for colonoscopy are associated with several digestive problems.

It is important to note that the methods used today for screening general population at risk of suffering for CRC or AA are associated with a high rate of false positives. Consequently, a high amount of unnecessary follow-up colonoscopies are nowadays performed.

The present invention offers a clear solution to the problems cited above because it is focused on an in vitro method for identifying or screening human subjects at risk of suffering from colorectal cancer or colorectal adenomas (particularly advanced colorectal adenomas). Since the method of the invention is preferably based on a liquid biopsy obtained from the patient (for example plasma, blood or serum), it is expected to improve compliance to colorectal cancer screening. Moreover, the method of the invention offers high sensitivity and specificity, which means that it is a strong and cost-effective method for the detection of both colorectal cancer and colorectal adenomas.

### BRIEF SUMMARY

The present invention refers to an in vitro method (hereinafter “method of the invention”) for the diagnosis or prognosis of colorectal cancer and/or a pre-cancerous stage thereof. It is preferably implemented in a liquid biopsy obtained from the patient, which is a minimally-invasive biological sample. The method of the invention offers high sensitivity and specificity, which means that it is a strong and cost-effective method for the detection of both colorectal cancer and/or a pre-cancerous stage thereof.

Since the method of the invention has higher sensitivity and specificity as compared to the method used today (FIT) for screening general population at risk of suffering from CRC or AA, it is associated with a lower percentage of false positives. Consequently, the method described in the present invention clearly helps in reducing the number of follow-up colonoscopies, thus improving the way that the patients are nowadays screened or diagnosed. Once the method of the invention is performed, if it is determined that the patients might be suffering from colorectal cancer and/or precancerous stage, the result is confirmed by colonoscopy. However, if it is not determined that the patient might be suffering from colorectal cancer and/or precancerous stage, there is no need to perform a colonoscopy and routine testing with the method of the invention is recommended.

Particularly, the first embodiment of the present invention refers to an in vitro method for the diagnosis of colorectal cancer and/or a pre-cancerous stage thereof, which comprises determining the methylation status of at least the gene LINC00473 in a liquid biopsy obtained from the patient, wherein a higher level of methylation of the gene LINC00473, as compared with a reference level of methylation of the gene LINC00473 measured in healthy subjects, is an indication that the subject is suffering from colorectal cancer and/or a pre-cancerous stage thereof.

The second embodiment of the invention refers to an in vitro method for the prognosis of colorectal cancer and/or a pre-cancerous stage thereof, which comprises determining the methylation status of at least the gene LINC00473 in a liquid biopsy obtained from the patient, wherein a higher level of methylation of the gene LINC00473, as compared with a reference level of methylation of the gene LINC00473 measured in the patient, is an indication that the patient has a poor prognosis.

The third embodiment of the present invention refers to the in vitro use of at least the methylation status of the gene LINC00473 for the diagnosis and/or prognosis of colorectal cancer and/or a pre-cancerous stage thereof.

The fourth embodiment of the present invention refers to the in vitro use of a kit comprising reagents for the determination of at least the methylation status of the gene LINC00473 for the diagnosis and/or prognosis of colorectal cancer and/or a pre-cancerous stage thereof.

In a preferred embodiment, the liquid biopsy is a sample of plasma, blood or serum. Plasma is particularly preferred.

In a preferred embodiment, the pre-cancerous stage is colorectal adenoma, preferably AA.

In a preferred embodiment, the methylation status of the gene LINC00473 is determined in at least a CpG of the promoter region.

In a preferred embodiment, the methylation status of the gene LINC00473 is determined in at least a CpG of the promoter region which is 3000 bp around the transcription start site (TSS). This means 1500 bp upstream or 1500 bp downstream of the TSS.

In a preferred embodiment, the methylation status of the gene LINC00473 is determined in at least a CpG of the promoter region located between the chromosomal positions Chr6: 166402081 and Chr6: 166402638.

In a preferred embodiment, the methylation status of the gene LINC00473 is determined in at least a CpG of the promoter region located at a chromosomal position selected from the group comprising: Chr6: 166402638, and/or Chr6: 166402474, and/or Chr6: 166402463, and/or Chr6: 166402457, and/or Chr6: 166402416, and/or Chr6: 166402379, and/or Chr6: 166402375, and/or Chr6: 166402364, and/or Chr6: 166402081, preferably a CpG selected from the group comprising: cg06545143, and/or cg08886973 and/or cg21306006.

In a preferred embodiment, the diagnosis of the colorectal cancer and/or a pre-cancerous stage thereof is confirmed by an image technique, preferably colonoscopy.

According to the method of the invention, after measuring the methylation status of the gene LINC00473, a score value is obtained and this score value is compared with a threshold value which defines the diagnostic rule. If this score value is higher than the threshold, then the corresponding sample is classified as a positive sample, which is an indication that the patient might be suffering from colorectal cancer and/or pre-cancerous stage thereof. The threshold value has been defined in order to optimize sensitivity and specificity values. Consequently, in a preferred embodiment, the method of the invention comprises: a) Measuring methylation status of the gene LINC00473, in a liquid biopsy obtained from the subject, b) processing the methylation values in order to obtain a risk score and c) wherein if a deviation or variation of the risk score value obtained is identified, as compared with a reference value, this is indicative that the subject is suffering from colorectal cancer and/or a pre-cancerous stage thereof.

The last embodiment of the present invention refers to a method for the diagnosis and treatment colorectal cancer or a pre-cancerous stage thereof, which comprises: a) obtaining a liquid biopsy from a human subject; b) detecting the methylation status of the gene LINC00473, and c) diagnosing the patient with colorectal cancer or a pre-cancerous stage thereof when a higher level of methylation of the gene LINC00473 is identified, as compared with a reference level of methylation of the gene LINC00473 measured in healthy subjects, and performing a colonoscopy to the patient. The colonoscopy may comprise the removal of the colorectal cancer or the polyps.

For the purpose of the present invention the following terms are defined:
LINC00473 (Long Intergenic Non-Protein Coding RNA 473) is an RNA Gene and is affiliated with the non-coding RNA class. It can be identified in public data bases as follows: HGNC: 21160. Entrez Gene: 90632. Ensambl ID: ENSG00000223414. UniProtKB: A8K010.
The term “colorectal cancer” is a medical condition characterized by cancer of cells of the intestinal tract below the small intestine (i.e., the large intestine (colon), including the cecum, ascending colon, transverse colon, descending colon, sigmoid colon, and rectum).
The expression “colorectal adenoma” refers to adenomas of the colon, also called adenomatous polyps, which is a benign and pre-cancerous stage of the colorectal cancer but still with high risk of progression to colorectal cancer.
The expression “advanced colorectal adenoma” refers to adenomas having a size of at least 10 mm or histologically having high grade dysplasia or a villous component higher than 20%; or serrated lesions having a size of at least 10 mm or dysplasia. For a more detailed explanation of the concept “advanced colorectal adenoma” please refer to the following scientific articles which pertain to the common general knowledge in the field of the invention: [Rex D K, Boland C R, Dominitz J A, Giardiello F M, Johnson D A, Kaltenbach T, Levin T R, Lieberman D, Robertson D J Colorectal Cancer Screening: Recommendations for Physicians and Patients From the U.S. Multi-Society Task Force on Colorectal Cancer. Gastroenterology. 2017 July; 153(1):307-323. doi: 10.1053/j.gastro.2017.05.013. Epub 2017 Jun. 9. PMID: 28600072][East J E, Atkin W S, Bateman A C, Clark S K, Dolwani S, Ket S N, Leedham S J, Phull P S, Rutter M D, Shepherd N A, Tomlinson I, Rees C J. British Society of Gastroenterology position statement on serrated polyps in the colon and rectum. Gut. 2017 July; 66(7):1181-1196. doi: 10.1136/gutjnl-2017-314005. Epub 2017 April 27. Review. PMID: 28450390].
The expression “liquid biopsy” refers to any sample of biologic fluids that may contain tumor-derived material. Particularly, a liquid biopsy is any sample of biologic fluids (for example: plasma, serum, blood, saliva, cerebrospinal fluid or urine) that may contain tumor-derived material, such as circulating tumor DNA.
The expression “minimally-invasive biological sample” refers to any sample which is taken from the body of the patient without the need of using harmful instruments, other than fine needles used for taking the blood from the patient, and consequently without being harmfully for the patient. Specifically, minimally-invasive biological sample refers in the present invention to: blood, serum, or plasma samples.
As used herein, the term “methylation” will be understood to mean the presence of a methyl group added by the action of a DNA methyl transferase enzyme to a cytosine base or bases in a region of nucleic acid, e.g., DNA.
Accordingly, the term, “methylation status” as used herein refers to the level of methylation measured in the patient which is being analyzed. The “methylation status” may be a higher/lower level of methylation as compared with the reference or control level.
As used herein, a “CpG dinucleotide,” “CpG methylation site” or equivalent, shall be taken to denote a cytosine linked to a guanine by a phosphodiester bond. CpG dinucleotides are targets for methylation of the cytosine residue and may reside within coding or non-coding nucleic acids. Non-coding nucleic acids are understood in the art to include introns, 5′-untranslated regions, 3′ untranslated regions, promoter regions of a genomic gene, or intergenic regions.
The expression “target CpG” refers to the specific CpG which is found to be differentially methylated in the present invention.
The expression “consecutive CpG” refers to CpGs which may be differentially methylated, and they can be found up to 1500 bp upstream or downstream of the transcription start site (TSS) (3000 bp around the TSS).
As used in the present invention “determining the methylation status of the gene” means that the method of the invention is performed by measuring the methylation status of any region of the gene, also comprising any regulatory sequence which is capable of increasing or decreasing the expression of said genes. Particularly, said regulatory sequence includes the promoter which is a region of DNA that initiates transcription of the gene. Promoters are located near the transcription start sites of genes, on the same strand, upstream or downstream on the DNA (towards the 5′ region of the sense strand).
As used herein, a “control, reference or baseline level of methylation” shall be understood to mean a level of methylation detected in a corresponding nucleic acid from a healthy patient. Thus, the patient is likely to suffer from CRC or AA, with a given sensitivity and specificity, if a “higher level of methylation” is measured in this patient as compared with a “control, reference or baseline level of methylation” measured in a healthy patient. A “reference value” can be a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Preferably, the person skilled in the art may compare the biomarker levels (or scores) obtained according to the method of the invention with a defined threshold value. Typically, the optimal sensitivity and specificity (and so the threshold value) can be determined using a Receiver Operating Characteristic (ROC) curve based on experimental data. For example, after determining the methylation level of the biomarkers in a group of reference, one can use algorithmic analysis for the statistic treatment of the measured methylation level of biomarkers in biological samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is good. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, preferably GraphPad Prism 6.
The expression “higher level of methylation” refers to a statistically significant increase in the relative amount of methylation of a nucleic acid, e.g., DNA, as compared with the amount of methylation measured in a subject/patient used as control/reference. Thus, in the present disclosure, the “higher level of methylation” is determined with reference to a baseline level represented by the methylation status of a given genomic region in a sample obtained from the subject or patient. For example, “higher level of methylation” may be at least 2% greater than the baseline level of methylation, for example at least 5% greater than the baseline level of methylation, or at least 10% greater than the baseline level of methylation, or at least 15% greater than the baseline level of methylation, or at least 20% greater than the baseline level of methylation, or at least 25% greater than the baseline level of methylation, or at least 30% greater than the baseline level of methylation, or at least 40% greater than the baseline level of methylation, or at least 50% greater than the baseline level of methylation, or at least 60% greater than the baseline level of methylation, or at least 70% greater than the baseline level of methylation, or at least 80% greater than the baseline level of methylation, or at least 90% greater than the baseline level of methylation.
By “comprising” it is meant including, but not limited to, whatever follows the word “comprising.” Thus, use of the term “comprising” indicates that the listed elements are required or mandatory, but that other elements are optional and may or may not be present.
By “consisting of” it is meant “including, and limited to,” whatever follows the phrase “consisting of” Thus, the phrase “consisting of” indicates that the listed elements are required or mandatory, and that no other elements may be present.

### BRIEF DESCRIPTION OF THE SEVERAL VIEWS OF THE DRAWINGS

FIG. 1. DNA methylation levels of LINC00473 promoter in colorectal tissue samples. In cohort 1 (A) and 2 (B), colorectal cancer patients showed significantly higher methylation levels of LINC00473 than non-tumor controls. The methylation levels of the LINC00473 promoter were analyzed (A) by Infinium Human Methylation 450K BeadChip (450K array), and (B) by pyrosequencing. DNA methylation data are expressed as β-values in A, and as % of methylation in B. The horizontal lines represent the median of the methylation levels. P, indicates the p-value analyzed by Mann-Whitney U test. Tumor, represents colorectal cancer.

FIG. 2. Receiver Operating Characteristics (ROC) curve for the methylation of LINC00473 promoter in colorectal tissue samples. The area under the ROC curve (AUC) in the Cohort 1 (A) and Cohort 2 (B) show the high diagnostic accuracy of the methylation of LINC00473 promoter to discriminate between colorectal cancer patients and non-tumor controls. P, indicates the p-value of the ROC curve. CI, is the confidence interval.

FIG. 3. DNA methylation levels of LINC00473 promoter in colorectal tissue samples from The Cancer Genome Atlas (TCGA) according to their clinical stage. All the stages of colorectal cancer patients showed significantly higher methylation levels of LINC00473 in (A) Cohort 1 (I: 0.55±0.023; II: 0.53±0.018; III: 0.45±0.028; IV: 0.47±0.031) and (B) Cohort 2 (I: 38.54%±7.67%; II: 34.31%±2.11%; III: 32.14%±2.84%; IV: 35.31%±3.95%) than their respective non-tumor controls (Cohort 1: 0.11±0.006; Cohort 2: 5.84%±0.78%). The methylation levels of LINC00473 were analyzed (A) by Infinium Human Methylation 450K BeadChip (450K array) and expressed as β-values, and (B) by pyrosequencing and expressed as % of methylation. Asterisks (*), indicate P<0.0001 respect to controls. The p-value was analyzed by Mann-Whitney U test. Tumor, represents colorectal cancer.

FIG. 4. DNA methylation levels of LINC00473 promoter in adenomas. Adenomas and colorectal cancer patients showed significantly higher methylation levels of LINC00473 than non-tumor controls. The methylation levels of the LINC00473 promoter were analyzed by pyrosequencing and expressed as % of methylation. The horizontal lines represent the median of the methylation levels. P, indicates the p-value analyzed by Mann-Whitney U test.

FIG. 5. Receiver Operating Characteristics (ROC) curve for the methylation of LINC00473 promoter in adenomas. The area under the ROC curve (AUC) in the Cohort 3 shows the high diagnostic accuracy of the methylation of LINC00473 promoter to discriminate between adenomas and non-tumor controls. P, indicates the p-value of the ROC curve. CI, is the confidence interval.

FIG. 6. DNA methylation levels of LINC00473 promoter in circulating plasma DNA of colorectal cancer patients and healthy controls. Colorectal cancer patients showed significantly higher methylation levels of LINC00473 than healthy controls. The methylation levels of LINC00473 were analyzed in triplicates by real-time PCR. The horizontal lines represent the median of the methylation levels. P, indicates the p-value analyzed by Mann-Whitney U test.

FIG. 7. Receiver Operating Characteristics (ROC) curve for the methylation of LINC00473 promoter in circulating plasma DNA of colorectal cancer patients respect to normal controls. The area under the ROC curve (AUC) shows the high diagnostic accuracy of the methylation of LINC00473 promoter to discriminate between colorectal cancer patients and healthy individuals. P, indicates the p-value of the ROC curve.

FIG. 8. Kaplan-Meier survival analysis for the methylation of LINC00473 promoter in colorectal cancer patients. Colorectal cancer patients were classified according to a higher or lower level of LINC00473 methylation respect to the median methylation level of the group (10% of methylation). Kaplan-survival analyses showed that high methylation levels of LINC00473 were significantly associated with shorter overall survival (OS) (P=0.0256). P, refers to the p-value of the Log-Rank test. H (High) indicates a methylation level >10%; and L (Low), a methylation level <10%.

FIG. 9. DNA methylation levels of LINC00473 promoter in circulating plasma DNA of advanced colorectal adenoma patients and healthy controls. Advanced colorectal adenoma patients showed significantly higher methylation levels of LINC00473 than healthy controls. The methylation levels of LINC00473 were analyzed in triplicates by real-time PCR. The horizontal lines represent the median of the methylation levels. P, indicates the p-value analyzed by Mann-Whitney U test.

FIG. 10. Receiver Operating Characteristics (ROC) curve for the methylation of LINC00473 promoter in circulating plasma DNA of advanced colorectal adenomas respect to normal controls. The area under the ROC curve (AUC) shows the high diagnostic accuracy of the methylation of LINC00473 promoter to discriminate between advanced colorectal adenomas and healthy controls. P, indicates the p-value of the ROC curve. CI, is the confidence interval.

FIG. 11. DNA methylation levels of LINC00473 promoter in advanced colorectal adenomas from tissues. Advanced colorectal adenomas showed significantly higher methylation levels of LINC00473 than non-tumor controls. The methylation levels of the LINC00473 promoter were analysed by pyrosequencing and expressed as % of methylation. The horizontal lines represent the median of the methylation levels. P, indicates the p-value analysed by Mann-Whitney U test.

FIG. 12. Receiver Operating Characteristics (ROC) curve for the methylation of LINC00473 promoter in advanced colorectal adenoma from tissues. The area under the ROC curve (AUC) shows the high diagnostic accuracy of the methylation of LINC00473 promoter to discriminate between advanced colorectal adenomas and non-tumor controls. P, indicates the p-value of the ROC curve. CI, is the confidence interval.

FIG. 13. DNA methylation levels of LINC00473 promoter in circulating plasma DNA of colorectal cancer by droplet digital PCR (ddPCR). Colorectal cancer patients showed significantly higher methylation levels of LINC00473 than non-tumor controls. The methylation levels of the LINC00473 promoter were analysed in triplicates by ddPCR. The horizontal lines represent the median of the methylation levels. P, indicates the p-value analysed by Mann-Whitney U test.

FIG. 14. Receiver Operating Characteristics (ROC) curve for the methylation of LINC00473 promoter in circulating plasma DNA of colorectal cancer patients respect to normal controls analysed by droplet digital PCR (ddPCR). The area under the ROC curve (AUC) shows the high diagnostic accuracy of the methylation of LINC00473 promoter to discriminate between colorectal cancer patients and healthy individuals. P, indicates the p-value of the ROC curve.

FIG. 15. DNA methylation levels of LINC00473 promoter in circulating plasma DNA of advanced colorectal adenomas by droplet digital PCR. Advanced colorectal adenoma patients showed significantly higher methylation levels of LINC00473 than healthy controls. The methylation levels of LINC00473 were analysed in triplicates by droplet digital PCR. The horizontal lines represent the median of the methylation levels and P indicates the p-value by analysed Mann-Whitney U test.

### DETAILED DESCRIPTION

### Example 1

### Methods

### Example 1.1

### Study Participants for Colorectal Tissue Analysis

We initially analyzed 3 independent cohorts (Cohort 1, Cohort 2, and Cohort 3) to study the DNA methylation of LINC00473 in colorectal tissues. Cohort 1 are represented by primary colorectal cancer patients (n=273) and non-tumor controls (n=38). The Cohort 2 includes 180 primary colorectal cancer patients and 93 non-tumor controls. And in the Cohort 3 there are 12 primary colorectal cancer patients, 12 adenomas and 12 non-tumor controls.

Moreover, for the purpose the present PCT application, we also analysed a new independent cohort (Cohort 4) to study the DNA methylation of LINC00473 in colorectal tissues, which includes 50 colorectal adenomas (comprising 20 confirmed advanced colorectal adenomas) and 10 non-tumor controls.

### Example 1.2

### Analysis of DNA Methylation Data of Colorectal Tissues by 450K Array

The results of DNA methylation of Cohort 1 were analyzed from the Infinium Human Methylation 450K BeadChip data (450K array) obtained in the public database The Cancer Genome Atlas (TCGA). The 450K array (Illumina) covers >450,000 CpG sites along the human genome, and methylation score of each CpG is represented as beta (β)-values that ranged between 0.0 (completely unmethylated) and 1.0 (completely methylated).

### Example 1.3

### Bisulfite Pyrosequencing Analysis of Colorectal Tissues

The DNA methylation status of LINC00473 promoter in the Cohorts 2, 3 and 4 were analyzed by bisulfite pyrosequencing. After bisulfite conversion of ˜500 ng of DNA with EZ-96 DNA Methylation kit (Zymo Research Corp.), the DNA methylation levels of LINC00473 promoter were analyzed with a PyroMark Q96 System (Qiagen) according to the manufacturer's instructions. CpG site methylation quantification was obtained using Pyro Q-CpG 1.0.9 (Qiagen). Primer sequences (Table 1) were designed with PyroMark Assay Design 2.0 (Qiagen). Table 1 shows the primers for methylation analyses of LINC00473 by pyrosequencing.

TABLE 1

| Primer sequence (5′-3′) | Primer size (nt) |
|---|---|
| F: SEQ ID NO: 1 | 27 |
| R: SEQ ID NO: 2 | 23 |
| S: SEQ ID NO: 3 | 22 |

F: Forward;
R: Reverse;
S: Sequencing;
nt: nucleotides

In a preferred embodiment, the primers used in the present invention, preferably the primer of SEQ ID NO: 2, are biotinylated by means of the incorporation of a molecule of biotin.

### Example 1.4

### Study Participants for Liquid Biopsy Analysis

For the analysis of LINC00473 in liquid biopsy of colorectal cancer, the study included 26 advanced colorectal cancer patients (stage III and IV) prospectively recruited at the time of diagnosis (baseline), before the beginning of the treatment and surgery. Demographic and clinical characteristics of the patients are represented in Table 2. Twenty-eight healthy controls were also included in the study. The disease status and staging of colorectal cancer patients was obtained by the medical oncologists of the Oncology Department at Complejo Hospitalario Universitario de Santiago de Compostela (CHUS). Table 2 shows clinical characteristics of the colorectal cancer patients.

TABLE 2

| Characteristics | Patients (n = 26) |
|---|---|
| Gender | |
| Male | 17 |
| Female | 9 |
| Age (years) | |
| Mean ± SD | 67 ± 10 |
| Stage | |
| III | 4 |
| IV | 22 |
| Histology | |
| Adenocarcinoma | 24 |
| Mucinous carcinoma | 2 |

SD: Standard deviation

For the analysis of LINC00473 in liquid biopsy of advanced colorectal adenoma, the study included 5 healthy controls and 12 advanced adenoma patients. All the individuals for liquid biopsy analysis were recruited at the Complejo Hospitalario Universitario de Santiago de Compostela (CHUS).

For the analysis of LINC00473 in liquid biopsy of colorectal cancer by droplet digital PCR (ddPCR), the study included 5 advanced colorectal cancer patients prospectively recruited at the time of diagnosis (baseline), before the beginning of the treatment and surgery. Ten advanced adenomas and 5 healthy controls were also included in the study. The patients and controls were obtained at Complejo Hospitalario Universitario de Santiago de Compostela (CHUS).

### Example 1.5

### Blood Sample Collection and Plasma Isolation

Blood samples were obtained by phlebotomy using collection tubes containing EDTA as anticoagulant. Plasma was isolated from blood samples within 2 h of collection with a first centrifugation of blood tubes (1,500 g, 10 min, 4° C.). Plasma was transferred to a 1.5 mL tubes and a second centrifugation was performed (15,000 g, 10 min, 4° C.). Plasma was transferred to new 1.5 mL tubes and stored at −80° C.

### Example 1.6

### Isolation of Circulating DNA from Plasma Samples

After defrosting the plasma samples, circulating DNA was isolated from 2-3 mL of plasma using the QIAamp® Circulating Nucleic Acid Kit (Qiagen) and the vacuum system QIAvac 24 Plus (Qiagen) following the manufacturer's recommendations. Circulating DNA was finally eluted in 75 μL of elution buffer and 1 μL of this elution was used to quantify the concentration of circulating DNA by the kit QuantiFluor® ONE dsDNA with the Quantus™ Fluorometer (Promega). After the quantification, circulating DNA was stored at −80° C.

### Example 1.7

### Bisulfite Conversion of Circulating DNA

For DNA bisulfite conversion, 15-50 ng of circulating DNA were used, and conversion was performed with EZ DNA Methylation-Lightning Kit (Zymo Research) according to manufacturer's recommendations following these thermocycling steps: 98° C. 8 min, 54° C. 60 min and 4° C. After column-based purification with the steps of binding, L-desulphonation and washing, bisulfite-converted DNA (bis-DNA) was eluted in 15 μL of elution buffer and stored at −80° C.

### Example 1.8

### DNA Methylation Analysis of The LINC00473 Promoter Region in Liquid Biopsy by Real-Time PCR

The methylation status of the promoter region of the long intergenic non-protein coding RNA 473 (LINC00473; previous symbol C6orfl76) was analyzed in circulating plasma DNA. The non-coding gene LINC00473 (NCBI ID: 90632; UCSC ID: uc063sul.1; Ensambl ID: ENSG00000223414; HGNC ID: HGNC:21160) is located in chromosome (chr) 6 at position 6q27, and has a CpG island containing its promoter region, which is located in chr6: 166401527-166402659. In addition, the promoter region of LINC00473 overlaps in the genome with the body of the long intergenic non-protein coding RNA 602 or LINC00602 (NCBI ID: 441177; UCSC ID: uc011egm.4; Ensambl ID: ENSG00000281832; HGNC ID: HGNC:43917).

The methylation levels of the promoter region of LINC00473 were determined by real-time PCR with a quantitative methylation-specific PCR assay (qMSP) in a StepOne Plus system (Applied Biosystems). Each reaction contained 2 uL of bisulfite-converted DNA (bis-DNA) as a template, 10 μl Power SYBR™ Green PCR Master Mix (ThermoFisher) and 150 nM each forward and reverse primers (Table 3) to detect methylation or unmethylation in a total volume of 20 μl. Reactions were performed in a MicroAmp™ Fast Optical 96-Well Reaction Plate (Applied Biosystems). Thermocycling conditions in StepOne Plus system were: 10 min at 95° C., followed by 50 cycles of 94° C. for 15 s and 60° C. for 30 s. Water was included as non-template control in each run to verify the absence of contamination during reactions. The colorectal cancer cell line HCT-116 and normal leukocytes (NL) were used in each analysis as positive controls for methylation and unmethylation, respectively. All the samples and controls were analyzed in triplicates. The threshold cycle (Ct) was determined for each reaction with the specific primers for methylation and unmethylation using the software StepOne Real-Time PCR (Applied Biosystems). The DNA methylation level of each sample was expressed as a percentage of methylation (%) according to the following calculation based on Cottrell et al. (2007):

Methylation (%)=100/[1+2\hat{ }(CT_{CG}−CT_{TG})]

In this formula, CT_{CG} represent the threshold cycle of the methylation status and CT_{TG} indicates the threshold cycle of the un-methylation status. With this calculation methylation levels can be between ranges of 0% to 100% of methylation. Table 3 shows the primers for methylation analyses of LINC00473 by real-time PCR.

TABLE 3

| | | Primer | Amplicon |
|---|---|---|---|
| Methylation status | Primer sequence (5′-3′) | size (nt) | size (bp) |
| Methylated | F: SEQ ID NO: 4 | 20 | 114 ⁽*⁾ |
| | R: SEQ ID NO: 5 | 20 | |
| Unmethylated | F: SEQ ID NO: 6 | 23 | 116 ^{(#)} |
| | R: SEQ ID NO: 7 | 23 | |

F: Forward; R: Reverse; nt: nucleotides; bp: base pairs.
⁽*⁾ Position: chr6: 166402364-166402477;
^{(#)} Position: chr6: 166402363-166402478.

The forward and reverse primers used for the methylation analysis of LINC00473 allows to detect the methylation status of 6 CpGs (Table 4) located at the promoter region of LINC00473. One of the CpGs (chr6: 166402416) contained in the sequence amplified (amplicon) by real-time PCR corresponds with the CpG termed cg08886973 according to the Infinium Human Methylation 450K and Infinium MethylationEPIC assays (Illumina), which are microarray assays able to detect the methylation status of more than 450,000 and 850,000 CpGs, respectively. These microarrays are also able to detect other CpGs from the LINC00473 promoter identified as cg06545143 (chr6: 166402638) and cg21306006 (chr6: 166402081) that are located, respectively, 162 nucleotides upstream and 282 nucleotides downstream the amplicon of LINC00473 analyzed in this study. The three CpGs included in this type of microarray assays (cg06545143, cg08886973, cg21306006) are located in the TSS1500 promoter region of LINC00473, which is the region of the promoter between 200 and 1500 nucleotides upstream its transcription start site (TSS). All the chromosomal positions previously indicated are according to the version GRCh37/hg19 of the UCSC Genome Browser from the University of California (https://genome.ucsc.edu/index.html). Table 4 shows the CpGs of LINC00473 that are detected in the methylation analyses by real-time PCR.

TABLE 4

| Methylation status | Primer | Chromosomal position |
|---|---|---|
| Methylated or | Forward | chr6: 166402474 |
| Unmethylated | | chr6: 166402463 |
| | | chr6: 166402457 |
| | Reverse | chr6: 166402379 |
| | | chr6: 166402375 |
| | | chr6: 166402364 |

### Example 1.9

### DNA Methylation Analysis of the LINC00473 Promoter Region in Liquid Biopsy by Droplet Digital PCR (DDPCR)

The methylation levels of the promoter region of LINC00473 were determined by droplet digital PCR (ddPCR) in a QX200 system (Bio-Rad). First, a multiplex preamplification reaction was performed using ˜2 ng of bisulfite-converted DNA (bis-DNA), 25 μl SsoAdvanced™ PreAmp Supermix (Bio-Rad), 0.5 μl of a custom Bio-Rad assay containing the forward and reverse primers and the probe for methylation (Bio-Rad) (Table 5), and 0.5 μl of a custom Bio-Rad assay containing the forward and reverse primers and the probe for unmethylation (Table 5), in a total volume of 50 μl. Final volume was completed with water. Reactions were performed in 0.2 ml PCR tubes (Axygen) on a ProFlex PCR System (Applied Biosystems): 3 min at 95° C., 10 cycles of 95° C. for 15 s and 56.2° C. for 4 min; and a final hold step of 4° C.

Next, a multiplex mix reaction was performed containing 2 uL of the pre-amplification product previously obtained, 11 μl ddPCR Supermix for Probes (No dUTP) (Bio-Rad), 2.2 μl of a custom Bio-Rad assay containing the forward and reverse primers and the probe for methylation (Table 5), and 2.2 μl of a custom Bio-Rad assay containing the forward and reverse primers and the probe for unmethylation (Bio-Rad) (Table 5), in a total volume of 22 μl. Final volume was completed with water. Then, 20 uL of each mix reaction and 70 μl of Droplet Generation oil (Bio-Rad) were transferred, respectively, to the sample and oil wells of a DG8™ Cartridge (BIO-RAD) and loaded into the QX200™ Droplet Generator (Bio-Rad). Drops were generated into the droplet well of the cartridge, transferred to a ddPCR 96-well plate (Bio-Rad) and loaded in a C1000 Touch Thermal Cycler (Bio-Rad): 10 min at 95° C., 40 cycles of 95° C. for 15 s and 56.2° C. for 30 s; 98° C. for 10 min and a final hold step of 4° C. The temperature ramp increment was 2.5° C./s for all steps. Then, the 96-well plate was read on a QX200™ Droplet Reader (Bio-Rad). Water was included as non-template control in each run to verify the absence of contamination during reactions. The colorectal cancer cell line HCT-116 and normal leukocytes (NL) were used in each analysis as positive controls for methylation and unmethylation, respectively. All controls were included in each plate with and without the preamplification step. All the samples and controls were analysed in triplicates. Analysis of the data was performed with the QuantaSoft software (Bio-Rad).

The DNA methylation level of each sample was expressed as a percentage of methylation (%) according to the following calculation:

Methylation (%)=[M/(U+M)]×100

In this formula, M (Methylation) represents the copies/l of the target DNA molecule for the methylation probe (FAM probe), and U (Unmethylation) indicates the copies/l of the target DNA molecule for the unmethylation probe (HEX probe). With this calculation methylation levels can be between ranges of 0% to 100% of methylation. Table 5 shows the sequences of primers and probes for methylation analyses of LINC00473 by ddPCR. Based on these sequences two custom Bio-Rad assays containing the primers and probe for methylation and unmethylation, respectively, were obtained from Bio-Rad according to manufacturer's recommendations: Primer to probe ratio: 1.8, primer concentration in final reaction 450 nM and probe concentration in final reaction 250 nM.

TABLE 5

| | Primer and probe | Primer/probe | Amplicon |
|---|---|---|---|
| Methylation status | sequence (5′-3′) | size (nt) | size (bp) |
| Methylated | F: SEQ ID NO: 4 | 20 | 114 ⁽*⁾ |
| | R: SEQ ID NO: 5 | 20 | |
| | P: FAM-SEQ ID NO: 8 | 20 | |
| Unmethylated | F: SEQ ID NO: 6 | 23 | 116 ^{(#)} |
| | R: SEQ ID NO: 7 | 23 | |
| | P: HEX- SEQ ID NO: 9 | 27 | |

F: Forward; R: Reverse; P: Probe; nt: nucleotides; bp: base pairs.
FAM: fluorescein;
HEX: hexachloro-fluoresceine
⁽*⁾ Position: chr6: 166402364-166402477;
^{(#)} Position: chr6: 166402363-166402478.

The forward and reverse primers, and the probe used for the methylation analysis of LINC00473 by ddPCR allows to detect the methylation status of 9 CpGs (Table 6) located at the promoter region of LINC00473. One of the CpGs (chr6: 166402416), which corresponds with the CpG termed cg08886973 according to the Infinium Human Methylation 450K and Infinium MethylationEPIC assays (Illumina), is contained in the sequence amplified (amplicon) and is detected with the methylated and unmethylated probe indicated in Table 5. All the CpGs positions of LINC00473 that are detected in the methylation analyses by ddPCR are represented in Table 6. All the chromosomal positions indicated in Table 6 are according to the version GRCh37/hg19 of the UCSC Genome Browser from the University of California (https://genome.ucsc.edu/index.html).

TABLE 6

| Methylation status | Primer | Chromosomal position |
|---|---|---|
| Methylated or | Forward | chr6: 166402474 |
| Unmethylated | | chr6: 166402463 |
| | | chr6: 166402457 |
| | Probe | chr6: 166402416 |
| | | chr6: 166402411 |
| | | chr6: 166402400 |
| | Reverse | chr6: 166402379 |
| | | chr6: 166402375 |
| | | chr6: 166402364 |

### Example 1.10

### Statistical Analyses

The Kolmogorov-Smirnov test was first used to evaluate the normality of the distribution of the data. Subsequently, the nonparametric Mann-Whitney U test was used for the comparison of the data. To assess the diagnostic accuracy of LINC00473 methylation to detect colorectal cancer a Receiver Operating Characteristic (ROC) curve was performed. The Youden index (J), which allows to obtain the cutoff point for methylation that provides the greatest combination of sensitivity and specificity, was calculated according to the formula: J=Sensitivity+Specificity-1 (Fluss et al., 2005). To measure the effectiveness of the diagnostic test, the positive (PPV) and negative predictive value (NPV) were calculated: PPV=true positive/(true positive+false positive); NPV=true negative/(true negative+false negative) (Hajian-Tilaki, 2013). To evaluate the survival analysis the Kaplan-Meier method was performed and the log-rank (Mantel-Cox) test was used to examine differences between the methylated and unmethylated group. The SPSS or GraphPad Prism 7.0 software was used for statistical analysis and graphic representation. All expressed β-values were calculated with two-tailed tests and were considered significant when p<0.05.

### Example 2. Results

### Example 2.1

### Tissue Samples

### Example 2.1.1

### The Methylation Analysis of the Promoter of LINC00473 in Tumor Tissues Allows Detecting Colorectal Cancer Patients

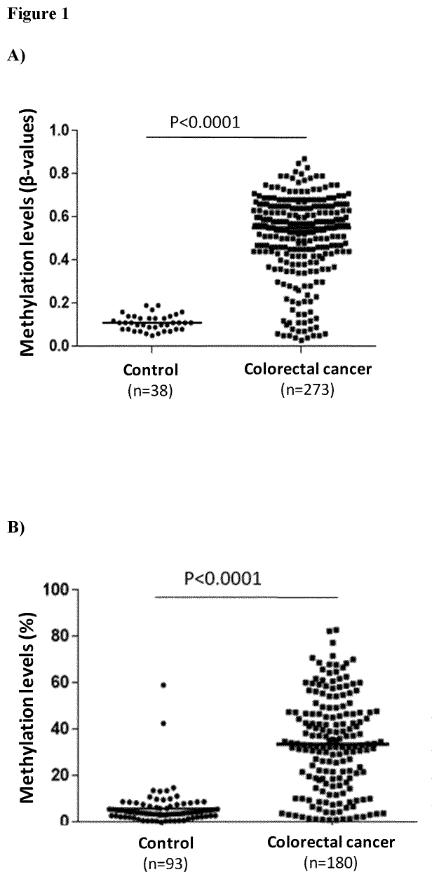

The DNA methylation levels of LINC00473 promoter region of colorectal tissue samples (273 colorectal cancer patients at stage I, II, III and IV; and 38 non-tumor controls) were obtained after the analysis by Infinium Human Methylation 450K BeadChip (450K array) from the international public database The Cancer Genome Atlas (TCGA) (Cohort 1). In this analysis the methylation data of 450K array are expressed as average beta (β)-values, which are defined from 0.0 to 1.0. We focused our analysis in the CpG of LINC00473 promoter region identified in the 450K array as cg08886973. This analysis showed significantly (p<0.0001) higher methylation levels in colorectal cancer patients (0.51±0.011) than in non-tumor controls (0.11±0.006) (FIG. 1A). Similar results were obtained when we analyzed the methylation status of the LINC00473 promoter in the Cohort 2 by bisulfite pyrosequencing (180 colorectal cancer patients: 33.93%±1.51%; 93 non-tumor controls: 5.84%±0.78%; p<0,0001) (FIG. 1). In this case, the methylation values obtained from pyrosequencing were expressed in percentage of methylation (%), which is defined from 0% to 100%. Altogether these data indicate that the methylation levels of the promoter region of LINC00473 analyzed in colorectal tissues are able to differentiate between non-tumor individuals and patients with colorectal cancer.

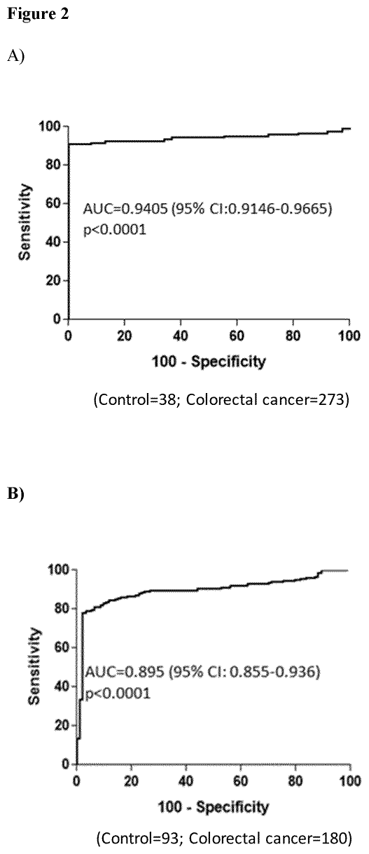

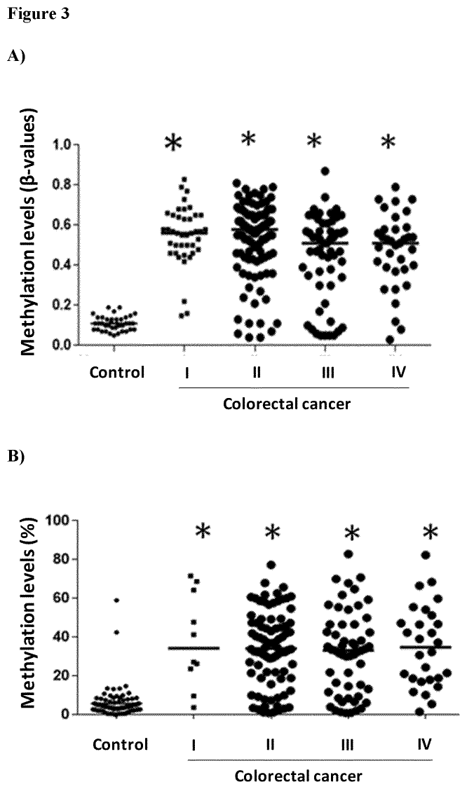

The analysis of the methylation levels of LINC00473 in the colorectal tissue samples from the Cohort 1 by receiver operating characteristic curve (ROC) showed a very high diagnostic accuracy to differentiate colorectal cancer patients (I, II, III and IV) from non-tumor controls with an area under the curve (AUC) of 0.94 (p<0.0001; CI 95%: 0.91-0.97) (FIG. 2A). Similar results were obtained from the Cohort 2 (AUC=0.90; p<0.0001; CI 95%: 0.86-0.94) (FIG. 2B). These results indicate that the analysis of the methylation status of the LINC00473 promoter region in colorectal tissue samples has a great diagnostic accuracy to identify colorectal cancer patients. Importantly, when we stratified the colorectal cancer patients according to their stage in the Cohort 1 (I: n=41; II: n=106; III: n=58; IV: n=35) and Cohort 2 (I: n=10; II: n=84; III: n=56; IV: n=28) (FIG. 3), the results also showed significantly (p<0.0001) higher methylation levels in each stage of colorectal cancer patients compared to non-tumor controls (Cohort 1, n=38; Cohort 2, n=93).

### Example 2.1.2

### The Methylation Analysis of the Promoter of LINC00473 in Colorectal Tissues Allows the Identification of Adenomas and Advanced Colorectal Adenomas

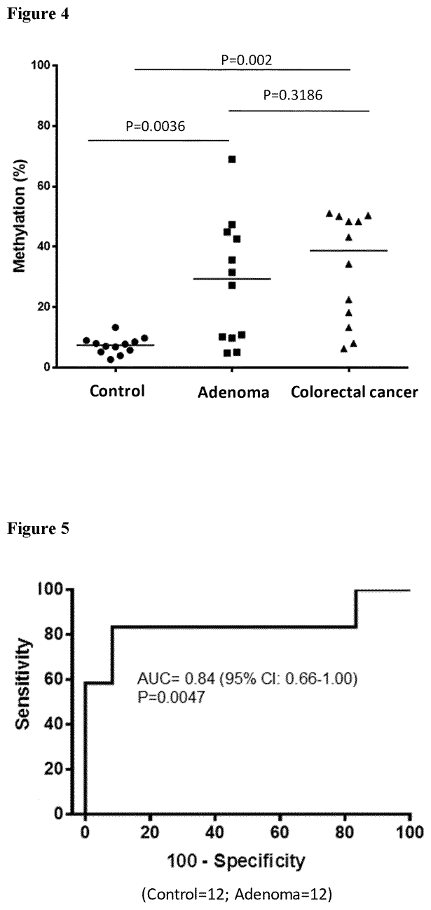

The DNA methylation levels of LINC00473 promoter region in colorectal tissues of 12 non-tumor controls, 12 adenomas and 12 colorectal cancer patients (Cohort 3) were analyzed by bisulfite pyrosequencing and expressed as % of methylation. These results showed significantly (p=0.0036) higher methylation levels in adenomas (28.21%±%) than in healthy controls (7.28%±0.81%) (FIG. 4). As expected, the colorectal cancer patients (32.83%±5.19%) also showed significantly (P=0.002) differences in methylation levels respect to the non-tumor controls. Importantly, the methylation levels of LINC00473 in adenomas were similar to colorectal cancer patients (P=0.3186). These data indicate that the methylation levels of the promoter region of LINC00473 analyzed in colorectal tissues are able to differentiate between non-tumor individuals and patients with adenomas, suggesting that the methylation status of the LINC00473 promoter can be useful for the early detection of colorectal cancer.

The analysis of the methylation levels of LINC00473 in the colorectal tissue samples from non-tumor and adenoma samples of Cohort 3 by receiver operating characteristic curve (ROC) showed a very high diagnostic accuracy to differentiate adenomas from non-tumor controls with an area under the curve (AUC) of 0.84 (p=0.0047; 95% CI: 0.66-1.00) (FIG. 5). These results indicate that the analysis of the methylation status of the LINC00473 promoter region in colorectal tissue samples has a great diagnostic accuracy to identify patients with colorectal adenomas, which are at risk of developing colorectal cancer.

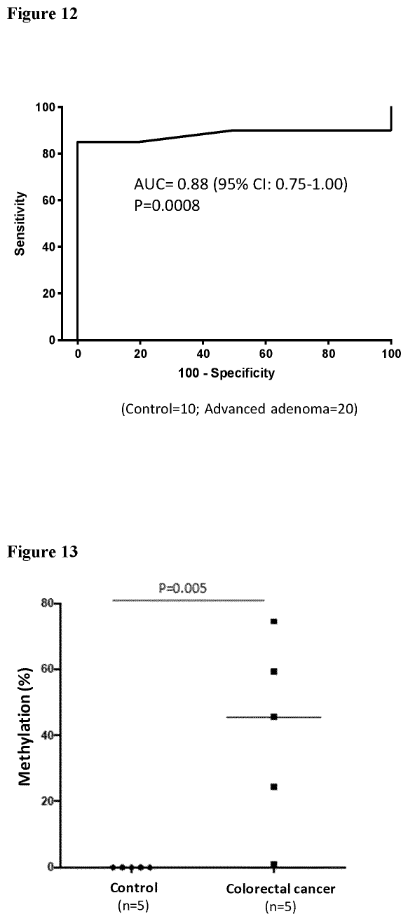

Moreover, in order to evaluate the methylation status of colorectal adenomas and advanced colorectal adenomas, we analysed the DNA methylation levels of LINC00473 promoter region by bisulfite pyrosequencing in a new independent cohort of tissue samples with 10 non-tumor controls and 50 adenomas, which included 20 confirmed advanced colorectal adenomas. This analysis showed significantly (p<0.0001) higher methylation levels in adenomas (20.96%±15.33%) than in healthy controls (5.70%±0.82%). Specifically, this analysis showed significantly (p<0.0003) higher methylation levels in advanced colorectal adenomas (26.35%±19.66%) than in controls (5.70%±0.82%) (FIG. 11). In fact, the receiver operating characteristic curve (ROC) analysis also showed a very high diagnostic accuracy to differentiate advanced colorectal adenomas from non-tumor controls with an AUC of 0.88 (p<0.0008; 95% CI: 0.75-1.00) (FIG. 12). These results confirm that the methylation levels of the promoter region of LINC00473 are useful for the detection of adenomas, specifically for the detection of advanced colorectal adenomas.

### Example 2.2

### Liquid Biopsy

### Example 2.2.1

### The Methylation Analysis of the Promoter of LINC00473 in Liquid Biopsy Detects Colorectal Cancer Patients

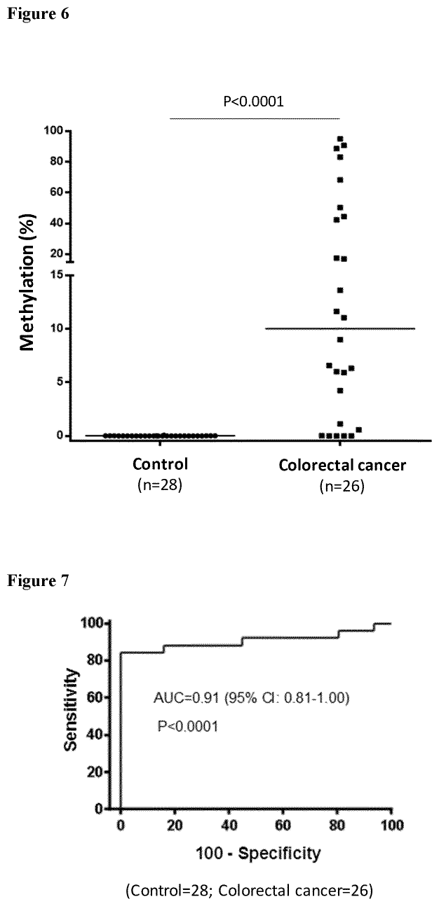

The DNA methylation levels (%) of LINC00473 promoter region in plasma of 28 controls and 26 colorectal cancer patients were analyzed by real-time PCR showing significantly (p<0.0001) higher methylation levels in colorectal cancer patients (25.87%±32.73%) than in healthy controls (0.003%±0.009%) (FIG. 6). Higher methylation levels of LINC00473 were detected in 21 out of 26 (81%) colorectal cancer patients respect to healthy individuals. These data indicate that the methylation levels of the promoter region of LINC00473 analyzed in liquid biopsy are able to differentiate between healthy individuals and patients with colorectal cancer.

The analysis of the methylation levels of LINC00473 in plasma by receiver operating characteristic curve (ROC) showed a very high diagnostic accuracy to differentiate colorectal cancer patients from healthy controls with an area under the curve (AUC) of 0.87 (p<0.0001; CI 95%: 0.76-0.99) (FIG. 7). Based on the ROC curve analysis and using the value of Youden's index, a methylation cutoff for LINC00473 in plasma of 0.30% was obtained, allowing the detection of colorectal cancer with a sensitivity of 81% (CI 95%: 61%-93%) and a specificity of 100% (CI 95%: 88-100) (Table 7). Using this methylation level cutoff, a positive predictive value (PPV) of 100% and a negative predictive value (NPV) of 85% was obtained for the detection of colorectal cancer. These results indicate that the non-invasive analysis in liquid biopsy of the methylation status of the LINC00473 promoter region has a great diagnostic accuracy to identify colorectal cancer patients. Table 7 shows the characteristics of the methylation analysis of LINC00473 promoter region as a diagnostic test for colorectal cancer detection.

TABLE 7

| Parameters | Percentage (%) |
|---|---|
| Sensitivity | 81 |
| Specificity | 100 |
| VPP | 100 |
| VPN | 85 |

VPP: Positive predictive value;
VPN: Negative predictive value

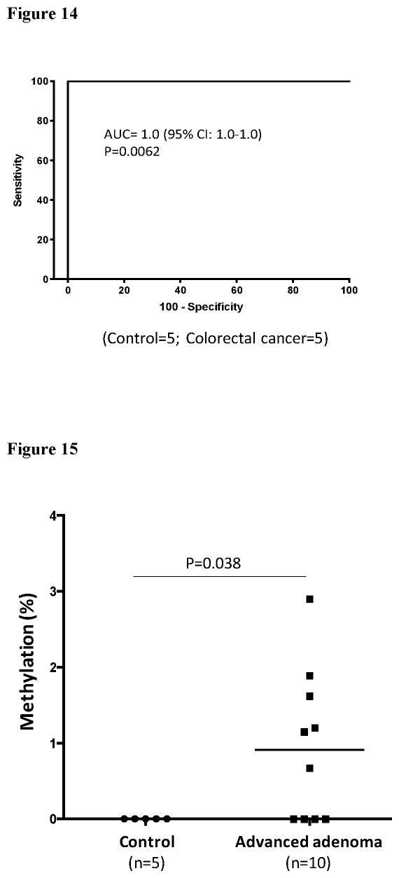

Moreover, DNA methylation levels (%) of LINC00473 promoter region in plasma of colorectal cancer patients were also evaluated using the droplet digital PCR (ddPCR) methodology. For this purpose, we used 5 controls and 5 colorectal cancer patients. These analysis showed significantly (p=0.005) higher methylation levels in colorectal cancer patients (41.19%±28.79%) than in healthy controls (0.0%±0.0%) (FIG. 13). Higher methylation levels of LINC00473 were detected in 5 out of 5 (100%) colorectal cancer patients respect to healthy individuals, indicating that the methylation levels of the promoter region of LINC00473 analyzed in liquid biopsy by ddPCR are also able to differentiate between healthy individuals and patients with colorectal cancer. In addition, the receiver operating characteristic curve (ROC) analysis showed that the methylation levels of LINC00473 in plasma obtained by ddPCR had a very high diagnostic accuracy to differentiate colorectal cancer patients from healthy controls with an area under the curve (AUC) of 1.0 (p<0.0062; CI 95%: 1.0-1.0) (FIG. 14). These results indicate that the non-invasive analysis in liquid biopsy of the methylation status of the LINC00473 promoter region by ddPCR has a great diagnostic accuracy to identify colorectal cancer patients.

### Example 2.2.2

### DNA Methylation Levels of the Promoter Region of LINC00473 in Liquid Biopsy Predict the Outcome of Colorectal Cancer Patients

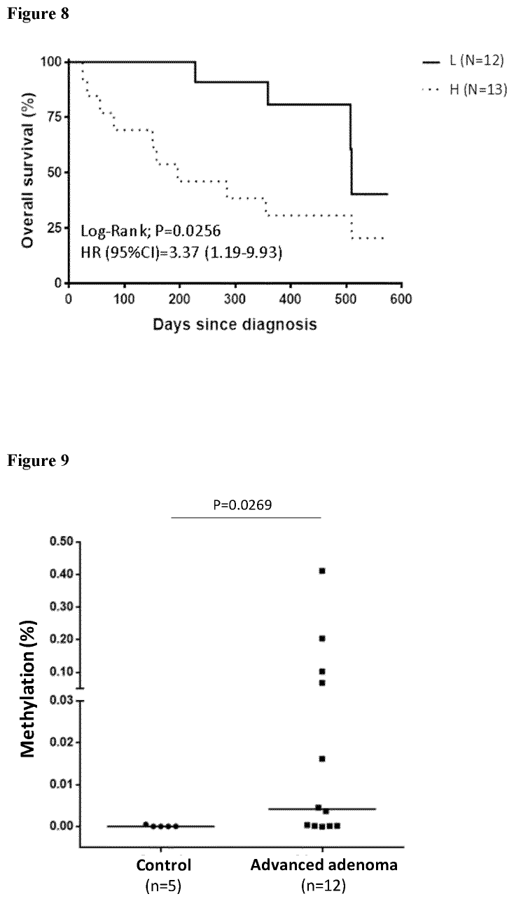

The clinical impact on survival of the methylation levels of LINC00473 promoter was studied in plasma samples of 25 colorectal cancer patients from Table 2 with available clinical data. Colorectal cancer patients were classified respect to the median methylation level of the group (methylation level of 10%) as presenting i) High level of methylation in LINC00473 (H: methylation level >10%) or ii) Low level of methylation in LINC00473 (L: methylation level <10%). According to this classification of the patients, Kaplan-Meir analysis showed that the presence of high levels of methylation in LINC00473 was significantly associated with shorter overall survival (OS) [hazard ratio (HR)=3.37, 95% confidence interval (CI)=1.19-9.93, P=0.0256] (FIG. 8). These results show that the methylation status of LINC00473 in liquid biopsy is able to predict the clinical outcome of colorectal cancer patients, suggesting that the analysis of LINC00473 methylation in plasma could be useful as a prognostic biomarker at the time of colorectal cancer diagnosis.

### Example 2.2.3

### The Methylation Analysis of the Promoter of LINC00473 in Liquid Biopsy Detects Advanced Colorectal Adenoma Patients

The DNA methylation levels (%) of LINC00473 promoter region in plasma of 5 healthy controls and 12 advanced colorectal adenoma patients were analyzed by real-time PCR showing significantly (p=0.0262) higher methylation levels in advanced colorectal adenoma patients (0.0676%±0.1195%) than in healthy controls (0.0001%±0.0002%) (FIG. 9). These data indicate that the methylation levels of the promoter region of LINC00473 analyzed in liquid biopsy are able to differentiate between healthy individuals and patients with advanced colorectal adenoma.

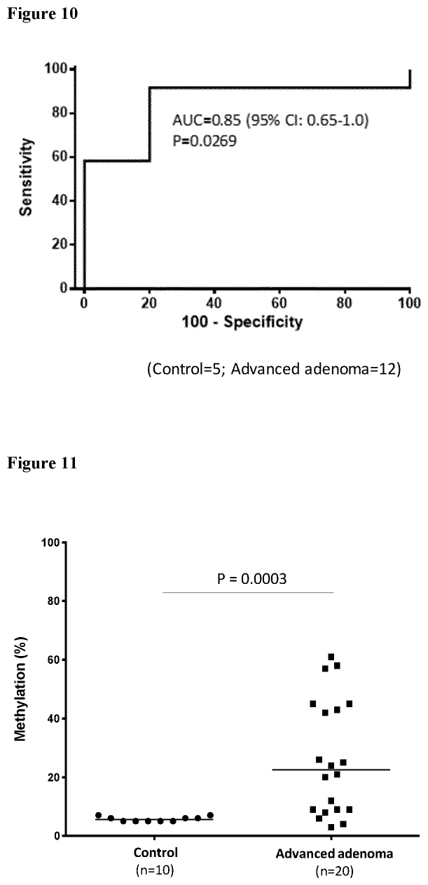

The analysis of the methylation levels of LINC00473 in plasma by receiver operating characteristic curve (ROC) showed a very high diagnostic accuracy to differentiate patients with advanced colorectal adenoma from healthy controls with an area under the curve (AUC) of 0.85 (p=0.00269; CI 95%: 0.65-1.0) (FIG. 10). Based on the ROC curve analysis, a methylation cutoff for LINC00473 in plasma of 0.000115 was obtained, allowing the detection of advanced adenoma with a sensitivity of 92% and a specificity of 80% (Table 8). Using this methylation level cutoff, a PPV of 92% and a NPV of 80% was obtained for the detection of advanced colorectal adenoma. These results indicate that the non-invasive analysis in liquid biopsy of the methylation status of the LINC00473 promoter region has a great diagnostic accuracy to identify patients with advanced colorectal adenomas. Table 8 shows the characteristics of the methylation analysis of LINC00473 promoter region as a diagnostic test for the detection of advanced colorectal adenoma.

TABLE 8

| Parameters | Percentage (%) |
|---|---|
| Sensitivity | 92 |
| Specificity | 80 |
| VPP | 92 |
| VPN | 80 |

VPP: Positive predictive value;
VPN: Negative predictive value

Moreover, DNA methylation levels (%) of LINC00473 promoter region in plasma were also evaluated by droplet digital PCR in 5 healthy controls and 10 advanced colorectal adenoma patients. These results showed significantly (p=0.038) higher methylation levels in advanced colorectal adenoma patients (0.94%±0.99%) than in healthy controls (0.0%±0.0%) (FIG. 15), indicating that the methylation levels of the promoter region of LINC00473 analysed in liquid biopsy by ddPCR are also able to differentiate between healthy individuals and patients with advanced colorectal adenoma.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-15. (canceled)

16. A method for determining the methylation status of the LINC00473 gene in a liquid biopsy obtained from a patient suspected of having a colorectal cancer and/or a pre-cancerous stage thereof, comprising:
performing a sequencing or amplification reaction of the LINC00473 gene;
determining the level of methylation of the LINC00473 gene based on results of the sequencing or amplification reaction; and
comparing the level of methylation of the LINC00473 gene to the level of methylation of the LINC00473 gene in a liquid biopsy obtained from a healthy patient.

17. The method of claim 16, comprising converting DNA in the liquid biopsy to bisulfite-converted DNA (bis-DNA), and performing the sequencing or amplification reaction of the LINC00473 gene in the bis-DNA.

18. The method of claim 16, wherein a higher level of methylation of the LINC00473 gene, as compared with the level of methylation of the LINC00473 gene measured in the healthy patient liquid biopsy, is an indication that the subject is suffering from colorectal cancer and/or a pre-cancerous stage thereof, or is an indication that the patient has a poor prognosis.

19. The method of claim 16, wherein the liquid biopsy is a sample of plasma, blood or serum.

20. The method of claim 16, wherein the pre-cancerous stage is colorectal adenoma,

21. The method of claim 16, wherein the pre-cancerous stage is advanced colorectal adenoma.

22. The method of claim 16, wherein the methylation status of the LINC00473 gene is determined in at least a CpG of the promoter region.

23. The method of claim 16, wherein the methylation status of the LINC00473 gene is determined in at least a CpG of the promoter region located between the chromosomal positions Chr6: 166402081 and Chr6: 166402638.

24. The method of claim 16, wherein the methylation status of the LINC00473 gene is determined in at least a CpG of the promoter region located at a chromosomal position selected from the group consisting of: Chr6: 166402638, Chr6: 166402474, Chr6: 166402463, Chr6: 166402457, Chr6: 166402416, Chr6: 166402379, Chr6: 166402375, Chr6: 166402364, and Chr6: 166402081.

25. The method of claim 16, wherein the methylation status of the LINC00473 gene is determined in at least a CpG selected from the group consisting of: cg06545143, cg08886973 and cg21306006.

26. The method of claim 16, further comprising performing an imaging technique to identify colorectal cancer and/or a pre-cancerous stage thereof in the patient.

27. The method of claim 16, wherein the imaging technique is a colonoscopy.

28. A method for treating a patient suspected of having colorectal cancer and/or a pre-cancerous stage thereof, comprising:
analyzing data for the methylation level of the LINC00473 gene from a liquid biopsy of the patient;
determining that the methylation level of the LINC00473 gene is higher than the methylation level of the LINC00473 gene in a healthy patient; and
performing an imaging technique on the patient.

29. The method of claim 28, wherein the imaging technique is a colonoscopy.

30. The method of claim 28, wherein the methylation level of the LINC00473 gene has been determined by a method comprising:
converting DNA in the liquid biopsy to bisulfite-converted DNA (bis-DNA);
performing a sequencing or amplification reaction of the LINC00473 gene in the bis-DNA; and
determining the level of methylation of the LINC00473 gene based on results of the sequencing or amplification reaction.

31. The method of claim 28, wherein the pre-cancerous stage is colorectal adenoma,

32. The method of claim 28, wherein the pre-cancerous stage is advanced colorectal adenoma.

33. A kit for analyzing a liquid biopsy of a patient suspected of having colorectal cancer and/or a pre-cancerous stage thereof, comprising:
a) primers for sequencing the LINC00473 gene in bisulfite converted DNA (bis-DNA) prepared from the liquid biopsy; and/or
b) primers for amplifying the LINC00473 gene in the bis-DNA, and optionally detectable probes for detecting amplification products of the LIN00473 gene, and
directions for determining the level of methylation of the LINC00473 gene based on results of a sequencing or amplification reaction, and comparing the level of methylation of the LINC00473 gene with the level of methylation of the LINC00473 gene in a liquid biopsy of a healthy patient.

34. The kit of claim 33, wherein the primers for sequencing the LINC00473 gene are selected from SEQ ID NOs: 1-3.

35. The kit of claim 34, wherein the primers for amplifying the LINC00473 gene are selected from SEQ ID NOs: 4-7, and the detectable probes are selected from SEQ ID NOs: 8-9.


SEQUENCE LISTING (9 sequences)

SEQ ID NO: 1 - DNA, 27 bp, Artificial sequence
  Primer sequence
ggggaggygataggtttggttttagtt

SEQ ID NO: 2 - DNA, 23 bp, Artificial sequence
  Primer sequence
acacaccaaaaatcttttctacc

SEQ ID NO: 3 - DNA, 22 bp, Artificial sequence
  Primer sequence
gataggtttggttttagttata

SEQ ID NO: 4 - DNA, 20 bp, Artificial sequence
  Primer sequence
tacggttttgggtcgttagc

SEQ ID NO: 5 - DNA, 20 bp, Artificial sequence
  Primer sequence
cgaaaccaacacgaacgata

SEQ ID NO: 6 - DNA, 23 bp, Artificial sequence
  Primer sequence
gtatggttttgggttgttagtgg

SEQ ID NO: 7 - DNA, 23 bp, Artificial sequence
  Primer sequence
ccaaaaccaacacaaacaataac

SEQ ID NO: 8 - DNA, 20 bp, Artificial sequence
  Probe sequence
taacgttgcgttatttgggc

SEQ ID NO: 9 - DNA, 27 bp, Artificial sequence
  Probe sequence
ttagttataatgttgtgttatttgggt